(19) United States
(12) Patent Application Publication
(10) Pub. No.: US 2020/0117176 A1
(43) Pub. Date: Apr. 16, 2020

(54) ROBOT ARM CONTROLLER AND ROBOT ARM SYSTEM

(71) Applicant: OLYMPUS CORPORATION, Tokyo (JP)

(72) Inventor: Tomoko GOCHO, Tokyo (JP)

(73) Assignee: OLYMPUS CORPORATION, Tokyo (JP)

(21) Appl. No.: 16/715,169

(22) Filed: Dec. 16, 2019

Related U.S. Application Data
(63) Continuation of application No. PCT/JP2017/023434, filed on Jun. 26, 2017.

(51) Int. Cl. G05B 19/42; A61B 1/00; G05B 19/404; B25J 9/16; B25J 3/00

(52) U.S. Cl. CPC G05B 19/42; A61B 1/00039; A61B 1/00043; G05B 2219/45118; B25J 9/16; B25J 3/00; G05B 19/404

(57) ABSTRACT

A robot arm controller controlling a treatment tool having a robot arm with an electrically driven joint has a driver configured to drive the treatment tool; a manipulator configured to accept a manipulation input for the treatment tool; and a processor. The processor is configured to: store a position of a distal end portion of the treatment tool as a teaching point when a teaching operation is detected from the manipulator, calculate an interpolation curve passing a plurality of teaching points and an interpolation point on the interpolation curve, control the driver to move the distal end portion of the treatment tool to the interpolation point, and store the position of the distal end portion of the treatment tool as a treatment point when a treatment operation is detected from the manipulator. The interpolation curve is updated so as to pass the plurality of teaching points and the treatment point.

This application is a continuation application based on a PCT International Application No. PCT/JP2017/023434, filed on Jun. 26, 2017. The content of the PCT International Application is incorporated herein by reference.

### BACKGROUND

### Field of the Invention

The present invention relates to a robot arm controller and a robot arm system having the robot arm controller.

### Description of Related Art

A treatment tool unit having a treatment portion such as a high-frequency knife and the like disposed at a distal end of a robot arm with multiple freedom degrees is used in a medical treatment such as a circumference dissection and the like.

For an operator during the treatment procedures, it is a stress to operate a robot arm with multi freedom degrees to properly determine a position of the treatment portion such as the high-frequency knife and the like disposed at the distal end and disposed the treatment portion at the determined position.

In Japanese Patent No. 5458769, an industrial robot arm configured to calculate an interpolation teaching point between a teaching point and another teaching point so as to generate a movement locus of the distal end thereof passing the teaching points and the interpolation teaching point is disclosed. The industrial robot arm can dispose the distal end thereof at a proper position by appropriately calculating the interpolation teaching point.

### SUMMARY

According to a first aspect of the present invention, a robot arm controller configured to control a treatment tool having a robot arm with an electrically driven joint, has a driver configured to drive the treatment tool; a manipulator configured to accept a manipulation input for the treatment tool; and a processor configured to control the treatment tool. The processor is configured to: store a position of a distal end portion of the treatment tool as a teaching point when a teaching operation is detected from the manipulator, calculate an interpolation curve passing a plurality of teaching points and an interpolation point on the interpolation curve, control the driver to move the distal end portion of the treatment tool to the interpolation point, and store the position of the distal end portion of the treatment tool as a treatment point when a treatment operation is detected from the manipulator. The interpolation curve is updated so as to pass the plurality of teaching points and the treatment point.

According to a second aspect of the present invention, in the robot arm controller according to the first aspect, the processor may be configured to be operated under a selected operation mode between a first mode and a second mode, the processor may be configured to detect the teaching operation from the manipulator in response to selecting the first mode, and the processor may be configured to calculate the interpolation point and control the distal end portion of the treatment tool to the interpolation point in response to selecting the second mode.

According to a third aspect of the present invention, in the robot arm controller according to the first aspect, the processor may be configured to cause the position of the distal end portion of the treatment tool to be adjustable according to the manipulation input from the manipulator while controlling the driver to move the distal end portion of the treatment tool to the interpolation point.

According to a fourth aspect of the present invention, a robot system has a treatment tool having a robot arm with an electrically driven joint; a console having the robot arm controller according to anyone of the first aspect to the third aspect, and an endoscope.

According to a fifth aspect of the present invention, in the robot system according to the fourth aspect, the treatment tool may be attached to the console to be relative movable with respect to the console, the manipulator may be configured to be able to make the treatment tool to relatively move with respect to the console, and the processor may be configured to store a relative movement amount of the treatment tool with respect to the console used while storing the teaching point and the processor may be configured to use the relative movement amount to calculate the interpolation point.

### BRIEF DESCRIPTION OF DRAWINGS

FIG. 1 is a view showing an overall configuration of a robot arm system according to a first embodiment of the present invention.

FIG. 2 is a view showing a configuration of a distal end of an overtube of the robot arm system.

FIG. 3 is a view showing a console of the robot arm system.

FIG. 4 is a schematic view showing a treatment tool unit of the robot arm system.

FIG. 5 is a view showing a manipulator of the console of the robot arm system.

FIG. 6 is a function block diagram of the manipulator of the robot arm system.

FIG. 7A is a view showing an overall configuration of a controller of the robot arm system.

FIG. 7B is a view showing an overall configuration of a controller of the robot arm system.

FIG. 8 is a view showing an example of a lesion portion which is dissected by the robot arm system.

FIG. 9 is a view showing the example of a lesion portion which is dissected by the robot arm system.

FIG. 10 is a view showing the example of a lesion portion which is dissected by the robot arm system.

FIG. 11 is a view showing the example of a lesion portion which is dissected by the robot arm system.

FIG. 12 is a view showing the example of a lesion portion which is dissected by the robot arm system.

FIG. 13 is a view showing the example of a lesion portion which is dissected by the robot arm system.

FIG. 14 is a view showing the example of a lesion portion which is dissected by the robot arm system.

FIG. 15 is a view showing the example of a lesion portion which is dissected by the robot arm system.

FIG. 16 is a flow chart showing a control flow of the controller of the robot arm system under a marking mode.

FIG. 17 is a flow chart showing a control flow of the controller of the robot arm system under an approach mode.

FIG. 18 is a flow chart showing a control flow under a teaching mode of a controller of a robot arm system according to a second embodiment of the present invention.

FIG. 19 is a flowchart showing a control flow under a marking mode of a controller of a robot arm system according to a third embodiment of the present invention.

FIG. 20 is a flow chart showing a control flow under an approach mode of the controller of the robot arm system according to the third embodiment of the present invention.

FIG. 21 is a flow chart showing a control flow under an approach mode of a controller of a robot system according to a modification example of the third embodiment of the present invention.

### DETAILED DESCRIPTION OF EMBODIMENTS

### First Embodiment

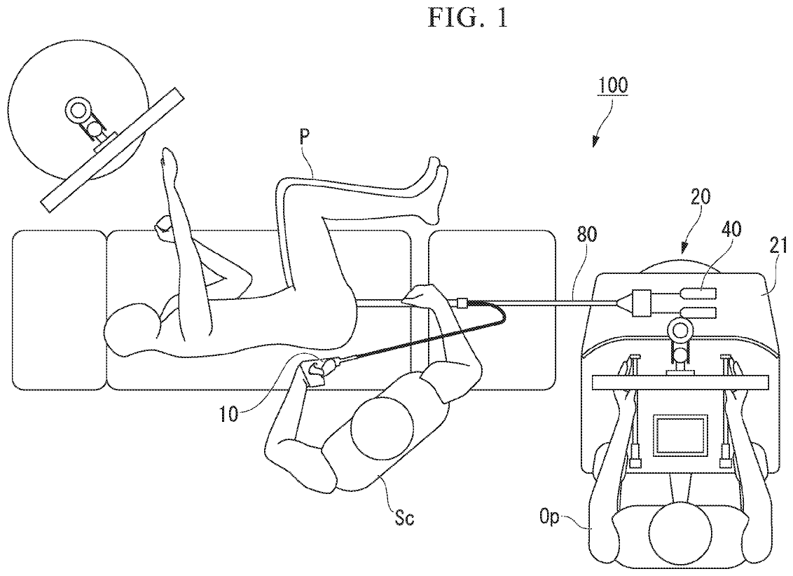

A first embodiment of the present invention will be described by referring to FIG. 1 to FIG. 17. In order to make the figures easy to view, dimension of each configuration element is properly adjusted.

FIG. 1 is a view showing an overall configuration of a robot arm system 100 of the present embodiment. The robot arm system 100 has an endoscope 10 configured to observe the inside of the body of a patient P, a manipulator (slave manipulator) 20 having a treatment tool unit (treatment tool) 40 for performing treatment inside the body of the patient P, and an overtube 80 through which the endoscope 10 and the manipulator 20 are inserted.

The endoscope 10 is a device for observing the inside of the body of the patient P and can be suitably configured by adopting various conventional configurations in consideration of the performance, the usage and the like.

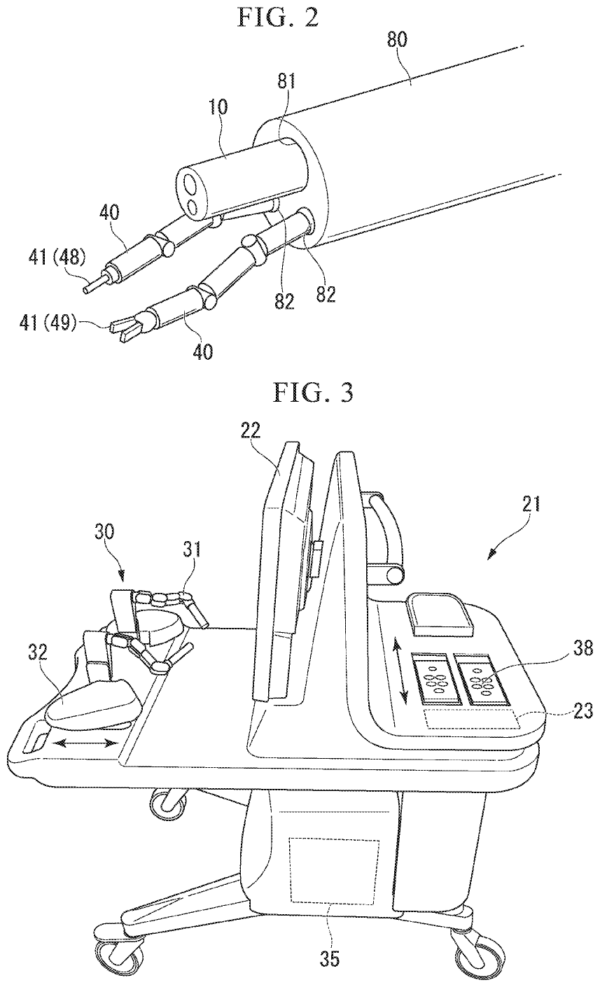

FIG. 2 is a view showing a configuration of the overtube 80.

As shown in FIG. 2, the overtube 80 has a first lumen 81 through which the endoscope 10 is inserted and a second lumen 82 through which the treatment tool unit 40 is inserted. With regard to the overtube 80, the overtube 80 can be suitably configured by adopting various conventional configurations in consideration of the dimension and the like. It is easy to approach a target portion for performing the treatment by using the overtube configured to have a bent portion at the distal end side thereof.

The manipulator 20 has a console 21 operated by an operator Op and a treatment tool unit 40 attached to the console 21.

FIG. 3 is a view showing the console 21.

The console 21 has an operation unit (manipulator, master manipulator) 30 to which the operator Op performs the operation input, a controller (robot arm controller) 35 configured to operate the treatment tool unit 40 according to the output from the operation unit 30, a motor unit 38 attached to the treatment tool unit 40, a monitor 22, and a stopper 23.

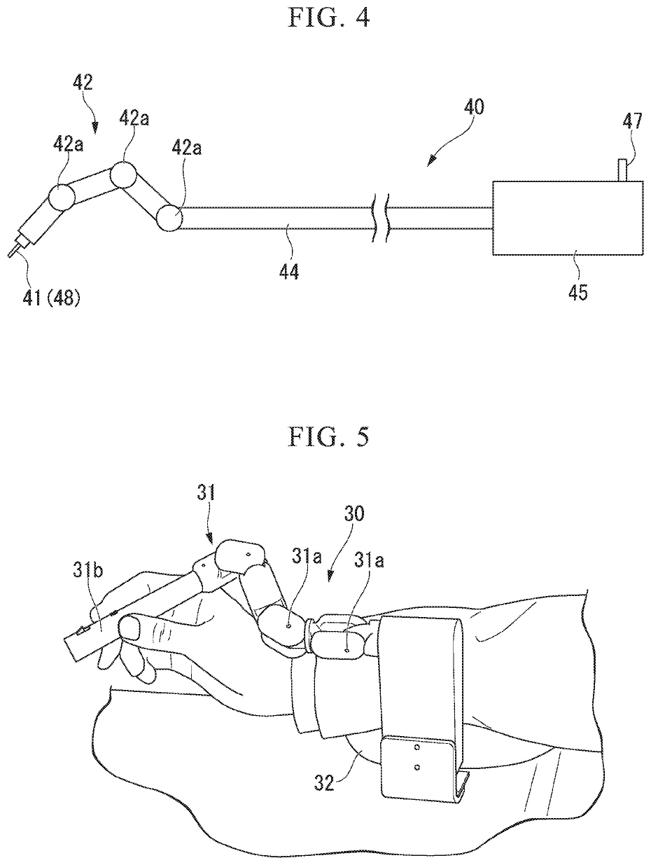

FIG. 4 is a schematic view showing the treatment tool unit 40.

The treatment tool unit 40 has an arm portion 43 including a treatment portion (distal end portion, end effector) 41 and an arm (robot arm) 42 to which the treatment portion 41 is attached, and an attachment portion 45 attached to the motor unit 38, wherein the attachment portion 45 is configured to transmit driving of the motor unit 38 so as to drive the treatment portion 41 and the arm 42. A flexible connection portion 44 having flexibility is formed in a region between the arm portion 43 and the attachment portion 45.

An electrical scalpel 48 as the treatment portion 41 is attached to the treatment tool unit 40 shown in FIG. 4. The electrical scalpel 48 is connected to an electrode 47 disposed at the attachment portion 45 by an electrical wiring. Electricity supplied from an external power supply controller (not shown) to the electrode 47 is transmitted to the electrical scalpel 48 via the electrical wiring.

The arm 42 has a plurality of joints. The plurality of joints 42 are connected to pulleys disposed in the attachment portion 45 by transmission members respectively.

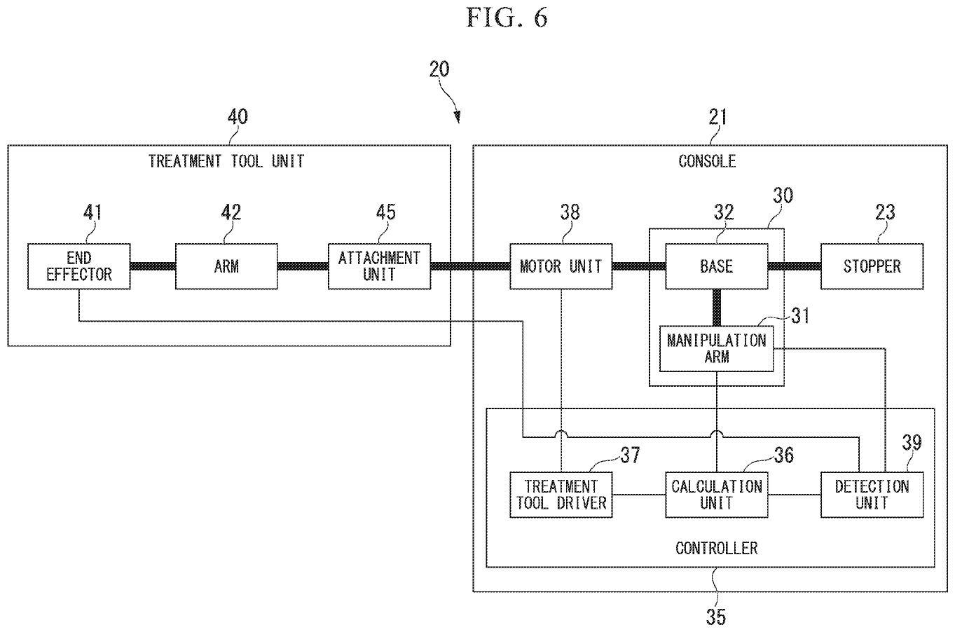

The pulleys are connected to an output shaft of the motor unit 38 so as to rotate due to an operation of the motor unit (see FIG. 6). Rotation shafts of the pulleys are supported by the attachment portion 45. Transmission members are wound on the pulleys and the transmission members advance and retract when the pulleys rotate so as to drive the plurality of joints 42a.

FIG. 5 is a view showing the operation unit 30 of the console 21.

The operation unit 30 has an operation arm 31 used to perform operation input with respect to the arm portion 43 and a base 32 to which the operation arm 31 is attached to.

The operation arm 31 is configured from a plurality of elongated members and end portions of adjacent two elongated members are connected by a joint 31a so as to be freely rotatable around a rotation axis.

The transmission members (not shown) are inserted through the operation arm 31 and the transmission members are connected to the joint 31a. A rotation angle of the rotation axis of the joint 31a is controlled by the advancement and retraction of the transmission members. The transmission members are disposed in each joint 31a.

The operation arm 31 and the arm 42 of the treatment tool unit 40 have the plurality of joints 31a and the plurality of joints 42a respectively. A number of the plurality of joints 31a is same with a number of the plurality of joints 42a, and the configuration of the rotation shaft of each joint is the same. A detection unit (not shown) such as an encoder and the like are provided at the joint 31a so as to be able to detect the rotation angle. Accordingly, when the operator Op operates the operation arm 31 as a master manipulator into an optional configuration, each joint 42a of the arm 42 as a slave manipulator is driven by the controller 35 to be in a configuration (similar configuration) corresponding to the configuration of the operation arm 31.

As shown in FIG. 5, a treatment operation unit 31b configured to move the treatment portion 41 is disposed at a distal end portion of the operation arm 31. A specific embodiment of the treatment operation unit 31b can be suitably determined according to the configuration of the treatment portion 41. For example, in a situation when the treatment portion 41 is a pair of grasping forceps 49, the treatment operation unit 31b may have a same configuration with that of the pair of grasping forceps 49. In a situation when the treatment portion 41 is a knife being used by applying electricity, the treatment operation unit 31b may be configured to have a switch bottom for tuning on/off the electricity.

The base 32 is attached so as to be relatively movable with respect to the console 21. When the base 32 is relatively moved with respect to the console 21, the motor unit 38 is relatively moved with respect to the console 21. Accordingly, it is possible to move the treatment tool unit 40 attached to the motor unit 38 relatively to the console 21.

FIG. 6 is a view showing a function block diagram of the manipulator 20 in a state in which the treatment tool unit 40 is attached to the console 21. In FIG. 6, the monitor 22 is omitted. In FIG. 6, the bold lines connecting the configurations indicate physical couplings capable of transmitting motive power, and fine lines connecting the configurations indicate logical couplings capable of performing signal transmission.

In the manipulator 20 according to the present embodiment, for example, the base 32 and the motor unit 38 are physically coupled by a belt, a chain, and the like. Accordingly, when the base 32 is moved relatively to the console 21, the motor unit 38 moves relatively with respect to the console 21 according to the base 32. At this time, the operation arm 31 moves together with the base 32, and the treatment tool unit 40 attached to the motor unit 38 moves together with the motor unit 38.

The monitor 22 is a device configured to display images acquired by the endoscope 10. The monitor 22 is configured by conventional display devices such as LCD display and the like.

The stopper 23 is physically coupled with the base 32. When the stopper 23 operates, the base 32 is held such that the base 32 does not move relatively with respect to the console 21.

The motor unit 38 is physically coupled with the arm portion 43 via the attachment portion 45. The motor unit 38 has a plurality of driving sources such as motors, and each joint 42a of the arm 42 is connected to each corresponding driving source by the transmission member via the pulley of the attachment portion 45.

The controller 35 is a device configured to control the manipulator 20. As shown in FIG. 6, the controller 35 has a control unit 36, a treatment tool driver 37, and a detection unit 39.

The control unit 36 has three operation modes as a manual mode, a marking mode (first mode), and an approach mode (second mode). The control unit 36 is configured to control the manipulator 20 according to a selected operation mode among the three modes.

The manual mode is an operation mode in which a scopist Sc operates the operation arm 31 to directly operate each joint 42a of the arm 42.

The marking mode is an operation mode in which the scopist Sc operates the operation arm 31 to directly operate each joint 42a of the arm 42 so as to teach part of dissection portion as the teaching point to the control unit 36.

The approach mode is an operation mode in which the scopist Sc may not operate the operation arm 31 and the control unit 36 operates each joint 42a of the arm 42 to determine the position of the treatment portion 41 and dispose the treatment portion 41 at the determined position.

The control unit 36 is configured by a device (computer) having hardware such as CPU (Central Processing Unit, processor), memory and the like which can execute program. The function of the control unit 36 is realizable as function of software by making the control unit 36 to read and execute program for controlling CPU.

Also, part or all of the functions of the control unit 36 may be realized by dedicated logic circuits rather than the function of software.

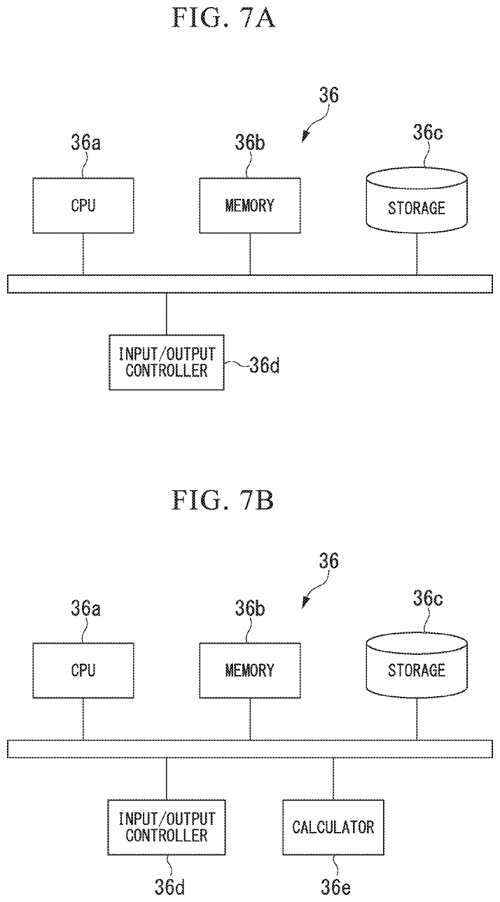

FIGS. 7A and 7B are views showing overall configurations of the control unit 36.

As shown in FIG. 7A, the control unit 36 has a CPU 36a, a memory 36b configured to be able to read program, a storage 36c, and an input/output control unit 36d. The program provided to the control unit 36 for controlling the operation of the controller 35 is read into the memory 36b and executed by the CPU (Central Processing Unit, processor) 36a.

The storage 36c is a non-volatile storage medium configured to store the program and necessary data. The storage 36c is configured by a ROM, a hard disk or the like. The program stored in the storage 36c is read into the memory 36b and executed by the CPU 36a.

The input/output control unit 36d is configured to receive input data from the operation arm 31, the detection unit 39, and the like and transfer the input data to the internal module of the control unit 36 such as the CPU 36a and the like. Also, the input/output control unit 36d is configured to generate a control signal for the treatment tool driver 37 and the like according to an instruction from the CPU 36a when the CPU 36a controls the treatment tool driver 37 and the like.

The control unit 36 may further have configurations for controlling the operation of the controller 35 besides the CPU 36a, the memory 36b, the storage 36c, and the input/output control unit 36d shown in FIG. 7A. For example, as shown in FIG. 7B, the control unit 36 may further have a calculation unit 36e configured to perform a part or all of specific calculation processing. The control unit 36 further has the calculation unit 36e so as to perform the specific calculation such as a matrix operation and the like rapidly.

The treatment tool driver 37 is configured to control the motor unit 38 according to the output of the control unit 36 so as to drive the treatment tool unit 40.

The treatment tool driver 37 is configured to drive the driving source such as the motors and the like provided in the motor unit 38 to cause the pulleys supported by the attachment portion 45 such that the treatment tool driver 37 can drive each joint 42a of the arm 42.

Also, the treatment tool driver 37 is configured to control the treatment portion 41. For example, in the situation when the electrical scalpel 48 is attached thereto as the treatment portion 41, the treatment tool driver 37 can control whether to supply the electricity to the electrical scalpel 48. In the situation when the grasping forceps 49 are attached thereto as the treatment portion 41, the treatment tool driver 37 can control the open/close operation of the grasping forceps 49.

The detection unit 39 is connected to the operation arm 31 and the treatment portion 41 and the detection unit 39 is configured to detect the movement of the operation arm 31 and the treatment portion 41. In the situation when the electrical scalpel 48 is attached thereto as the treatment portion 41, the detection unit 39 can detect the operation of the operator to operate the treatment operation unit 31b of the operation arm 31 for supplying the electricity to the electrical scalpel 48.

The detection unit 39 can further detect whether the electrical scalpel 48 actually comes in contact with the lesion portion according to the impedance value measured from the electrical circuit of the electrical scalpel 48 when the electricity is supplied to the electrical scalpel 48 (Fire). A predetermined value of the impedance is set in the detection unit 39, and the predetermined value is set between an impedance value when the electricity is supplied thereto at a state in which the electrical scalpel 48 comes in contact with the lesion portion and an impedance value when the electricity is supplied thereto at a state in which the electrical scalpel 48 does not come in contact with the lesion portion. When the electricity is supplied to the electrical scalpel 48 (Fire), if the impedance value measured from the electrical circuit of the electrical scalpel 48 is equal to or lower than the predetermined value, the state in which the electricity is supplied thereto in the state in which the electrical scalpel 48 comes in contact with the lesion portion can be detected.

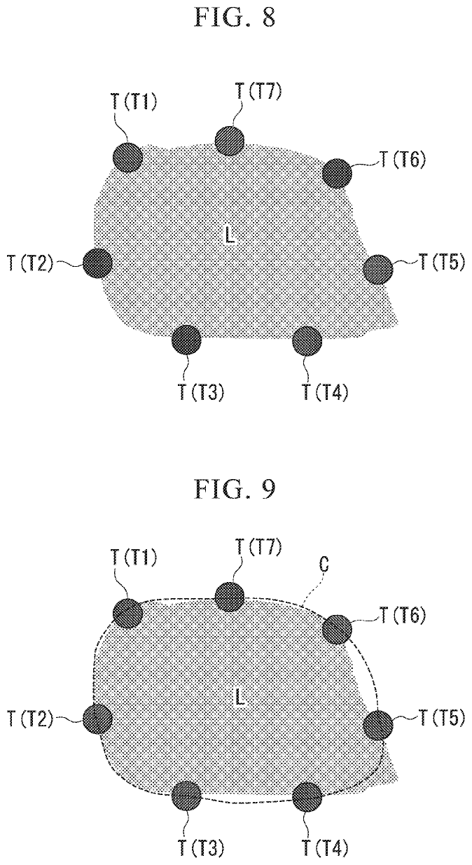

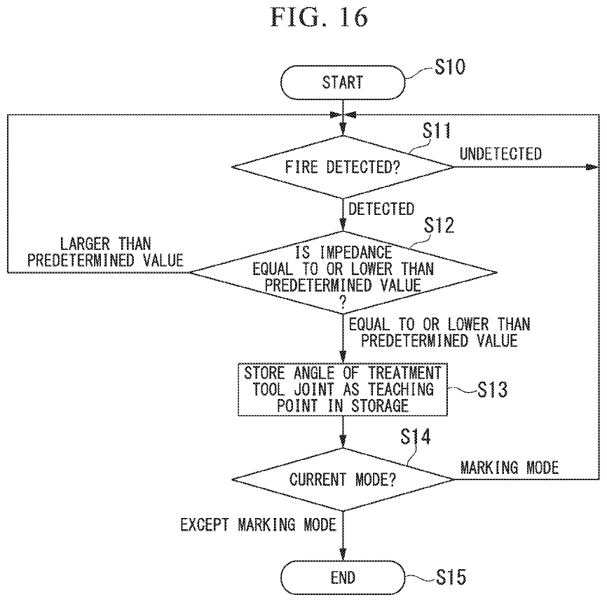

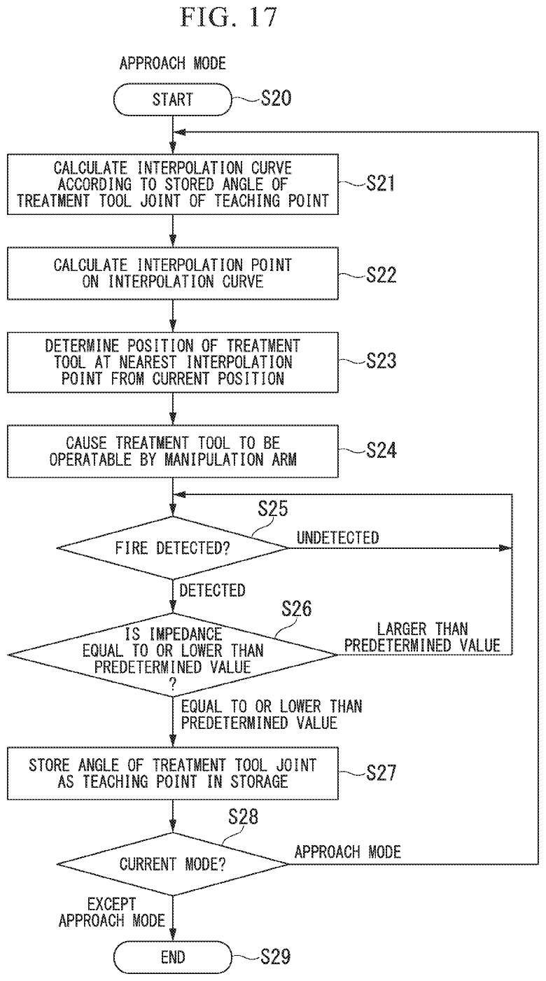

An operation of using the robot arm system 100 having the above-described configuration will be described by referring to FIG. 8 to FIG. 17. FIG. 8 to FIG. 15 are views showing the lesion portion L which is dissected by the robot arm system 100. FIG. 16 is a control flow chart of the control unit 36 in the marking mode. FIG. 17 is a control flow chart of the control unit 36 in the approach mode.

As shown in FIG. 1, the robot arm system 100 is operated by at least two operators such as the operator Op operating the console 21 and the scopist Sc operating the overtube 80 and the endoscope 10.

As preparation procedures, as shown in FIG. 1, the scopist Sc inserts the endoscope 10 into the first lumen 81 of the overtube 80. Also, the scopist inserts the treatment tool unit 40 into the second lumen 82 of the overtube 80.

The scopist Sc inserts the overtube 80 through which the endoscope 10 and the treatment tool unit 40 are inserted into the anus of the patient P. Subsequently, the scopist Sc advances the overtube 80 in the large intestine while observing the image acquired by the endoscope 10 to introduce the overtube 80 through which the endoscope 10 is inserted to the vicinity of the target portion.

Subsequently, the scopist Sc protrudes the endoscope 10 from the overtube 80 and performs bending operations if necessary, to secure the visual field for the treatment with respect to the target portion. Thus, the preparation procedures are finished.

After the preparation procedures are finished, the operator Op attaches the attachment portion 45 of the treatment tool unit 40 to the motor unit 38 of the console 21. When the operator Op grasps the operation arm 31 and moves the base 32 toward his/her front side, the motor unit 38 and the attachment unit 45 are interlocked with each other that the treatment tool unit 40 moves toward the distal end side of the overtube 80. As a result, as shown in FIG. 2, the arm portion 43 protrudes from the overtube 80.

After the arm portion 43 protrudes from the overtube 80, the operator Op performs a predetermined input to the operation unit 30 so as to associate the operation arm 31 with the arm portion 43. According to the predetermined input, an associating instruction is output from the operation unit 30 to the control unit 36.

The control unit 36 accepted the associating instruction acquires information showing the state of each joint 31a in the operation arm 31 and information showing the state of each joint 42a in the arm 42. Subsequently, the control unit 36 calculates the movement amount necessary for each joint 42a so as to make the arm 42 to deform a shape similar to that of the operation arm 31 according to the acquired information, and the control unit 36 transmits the movement amount to the treatment tool driver 37.

The treatment tool driver 37 generates a driving signal for driving each joint 42a according to the received necessary movement amount of each joint 42a and transmits the driving signal to the motor unit 38. The motor unit 38 is driven to move each joint 42a such that the association is finished when the arm 42 and the operation arm 31 are in the similar shapes which are substantially the same.

After the association is finished, the processing same with the association is repeated by a predetermined period (for example, one millisecond). Accordingly, the arm portion 43 attached to the treatment portion 41 is controlled to maintain the similar shape with that of the operation arm 31.

After the association is finished, the operation mode of the control unit 36 is set to the manual mode. The operator Op can suitably operate the operation arm 31 and the treatment operation unit 31b to perform the desirable operation with respect to the target portion while confirming the video of the target portion displayed on the monitor 22. The operator Op operates the operation arm 31 and the scopist operates the endoscope 10 to move the treatment portion 41 and the endoscope 10 to the position possible to perform the treatment with respect to the target portion.

In the situation in which the operator Op performs the dissection with respect the lesion portion, the operator Op or the scopist Sc sets the operation mode of the control unit 36 to the marking mode. The hatched part in FIGS. 8-15 show the lesion part L dissected by the robot arm system 100. Hereinafter, the control flow of the control unit 36 in the marking mode will be described by referring to FIG. 16.

As shown in FIG. 16, when the operation mode of the control unit 36 is changed to the marking mode, the control unit 36 starts the control of the marking mode (Step S10). Next, the control unit 36 executes Step S11.

As shown in FIG. 8, the operator Op cauterizes several points around the lesion portion L as the teaching points T. The operator Op operates the operation arm 31 to move the electrical scalpel 48 to the part around the lesion portion L and makes the electrical scalpel 48 to contact with the part around the lesion portion L. Furthermore, the operator Op supplies the electricity (Fire) to the electrical scalpel 48 to actually cauterize the teaching points T.

In Step S11, as shown in FIG. 16, the control unit 36 confirms whether the signal indicating that the treatment operation portion 31b is operated and the electrical scalpel 48 is supplied with the electricity (Fire) is input from the detection unit 39. In the situation in which the signal is input, the control unit 36 executes Step S12. In the situation in which the signal is not input, the control unit 36 keeps in standby until the signal indicating that the electrical scalpel 48 is supplied with the electricity (Fire) is input.

In Step S12, as shown in FIG. 16, the control unit 36 determines whether an impedance value measure from the electrical circuit of the electrical scalpel 48 is equal to or smaller than a predetermined value. In the situation in which the impedance value measure from the electrical circuit of the electrical scalpel 48 is equal to or smaller than the predetermined value, the electrical scalpel 48 is supplied with the electricity (Fire) and the electrical scalpel 48 is in contact with the part around the lesion portion L to actually cauterizes the part around the lesion portion L. In the situation, the control unit 36 executes Step S13 subsequently.

In the situation in which the impedance value measure from the electrical circuit of the electrical scalpel 48 is larger than the predetermined value, the control unit 36 returns to Step S11 to be in standby for the electrical scalpel 48 to be supplied with the electricity (Fire).

In Step S12, in the situation in which control unit 36 determines that the impedance value measure from the electrical circuit of the electrical scalpel 48 is equal to or smaller than the predetermined value, the detection unit 39 and the control unit 36 determines that the operator Op has performed the teaching operation of the teaching points T according to the operation of the operation portion 30.

Also, the detection unit 39 and the control unit 36 may pass Step S12 and determine that the operator Op has performed the teaching operation of the teaching points T according to that the input of the signal indicating that the electricity is supplied (Fire) to the electrical scalpel 48 is detected.

In Step S13, as shown in FIG. 16, the control unit 36 records the position of the treatment portion (distal end portion) 41 of the treatment tool unit 40 as the teaching points T in the memory 36b and the storage 36c when the electrical scalpel 48 is supplied with the electricity (Fire). The record target in the present embodiment is an angle of the joint 42a of the treatment tool unit 40. The position of the treatment portion (distal end portion) 41 of the treatment tool unit 40 can be calculated if the angle of the joint 42a is recorded in advance. The angle of the joint 42a of the treatment tool unit 40 may be calculated from the control information of the joint 42a, and may be acquired from the encoder provided at the joint 31a of the operation arm 31.

The record target may not be the angle of the joint 42a of the treatment tool unit 40. The record target may be three-dimensional coordinates of the electrical scalpel 48 calculated from the angle.

In Step S14, as shown in FIG. 16, the control unit 36 determines that the operation mode is the marking mode. In the situation in which the operation mode is still the marking mode, the control unit 36 executes Step S11 and wait for the teaching of the next teaching points T.

In the situation in which the operation mode is not the marking mode, the control unit 36 executes Step S15 subsequently and the control of the marking mode is finished.

The operator Op or the scopist Sc can make the control unit 36 to finish the control of the marking mode by changing the operation mode of the control unit 36 to another mode except the marking mode.

As shown in FIG. 8, the operator Op cauterizes a plurality of teaching points T surrounding the lesion portion L. A number of the teaching points T and an interval between the teaching points T are not particularly limited. The teaching points T may be any embodiment only if the teaching points T are formed to surround the lesion portion L. After marking the plurality of teaching points T is finished, the operator Op or the scopist Sc may change the operation mode of the control unit 36 to another one except the marking mode to finish the control of the marking mode by the control unit 36. In the example shown in FIG. 8, after the operator Op cauterizes seven teaching points T (T1-T7), the marking mode is terminated.

The operator Op or the scopist Sc sets the operation mode of the control unit 36 to the approach mode. Hereinafter, the control flow of the control unit 36 in the approach mode will be described following FIG. 17.

As shown in FIG. 17, when the operation mode of the control unit 36 is changed to the approach mode, the control unit 36 starts the control of the approach mode (Step S20). The control unit 36 deactivates the input operation of the operation arm 31. Accordingly, the operator Op cannot operate the operation arm 31 to operate the joints 42a of the arm 42 of the treatment tool unit 40 and the like. Next, the control unit 36a executes Step S21.

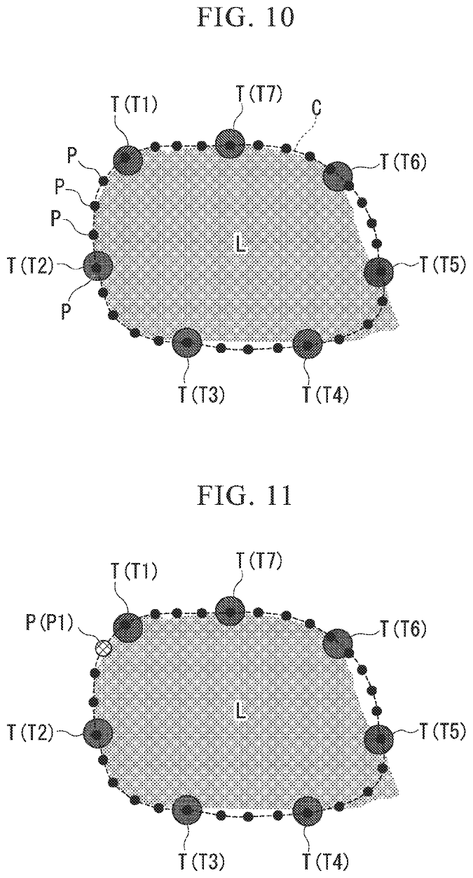

As shown in FIG. 17, in Step S21, the control unit 36 seeks an interpolation curve C passing the teaching points T shown as FIG. 9 according to the information of the recorded plurality of teaching points T. In Step S13, in situation in which the information recorded by the control unit 36 is the angles of the joints 42a of the treatment tool unit 40, the control unit 36 seeks the three-dimensional coordinates of the teaching points T according to the angles.

The control unit 36 seeks the interpolation curve C pas sing the plurality of teaching points (T1-T7) shown as FIG. 9 according to the three-dimensional coordinates of the plurality of teaching points (T1-T7). A conventional method for calculating an interpolation curve such as the Bezier curve can be used as a method for calculating the interpolation curve C. Next, the control unit 36a executes Step S22.

In Step S22, as shown in FIG. 17, the control unit 36 disposes interpolation points P on the interpolation curve C. The interpolation points P are disposed with a predetermined interval therebetween. As shown in FIG. 10, the interpolation points P may be superimposed with the teaching points T. Also, the predetermined interval may be changed by the operator Op or the scopist Sc. The interpolation points P may be disposed on the interpolation curve C by irregular intervals. Subsequently, the control unit 36 executes Step S23.

It is preferable to dispose the interpolation points P in a configuration such that the lesion portion L can be removed if all of the interpolation points P are cauterized.

Here, a minimum interval among the interpolation points P is preferable to be the minimum movement amount of the robot arm.

In Step S23, as shown in FIG. 17, the control unit 36 calculates the current three-dimensional coordinates of the electrical scalpel 48 and selects the nearest interpolation point P with respect to the electrical scalpel 48 from the plurality of interpolation points P. Among the plurality of interpolation points P as shown in FIG. 11 as an example, the interpolation point P1 it assumed to be the currently nearest interpolation point P with respect to the electrical scalpel 48.

Next, the control unit 36 executes Step S24.

In Step S24, as shown in FIG. 17, the control unit 36 activate the operation input of the operation arm 31. The operator Op can operate the operation arm 31 to operate the joints 42a of the arm 42 of the treatment tool unit 40 and the like.

Next, the control unit 36 executes Step S25.

In the situation in which the position of the electrical scalpel 48 disposed by the control unit 36 is the position of the scheduled dissection point that is described by the operator Op, the operator Op does not adjust the position and operates the electrical scalpel 48 to come in contact with the interpolation point P1.

On the other hand, in the situation in which the position of the electrical scalpel 48 disposed by the control unit 36 is not the position of the scheduled dissection point that is described by the operator Op, the operator Op operates the operation arm 31 to slightly adjust the position, and the operator Op makes the electrical scalpel 48 after the slight adjustment to come in contact with the desired scheduled dissection point.

In Step S25, as shown in FIG. 17, in the same manner with that in Step S11, the control unit 36 confirms whether the signal indicating that the electrical scalpel 48 is supplied with the electricity (Fire) is input from the detection unit 39. In the situation in which the signal is input, the control unit 36 executes Step S26 subsequently. In the situation in which the signal is not input, the control unit 36 keeps in standby for the input of the signal indicating that the electrical scalpel 48 is supplied with the electricity (Fire).

In Step S26, as shown in FIG. 17, in the same manner with that in Step S12, the control unit 36 determines whether then impedance value measure from the electrical circuit of the electrical scalpel 48 is equal to or smaller than a predetermined value. In the situation in which the impedance value measure from the electrical circuit of the electrical scalpel 48 is equal to or smaller than the predetermined value, the control unit 36 executes Step S27 subsequently.

In the situation in which the impedance value measure from the electrical circuit of the electrical scalpel 48 is larger than the predetermined value, the control unit 36 returns to Step S25 to be in standby for the electrical scalpel 48 to be supplied with the electricity (Fire).

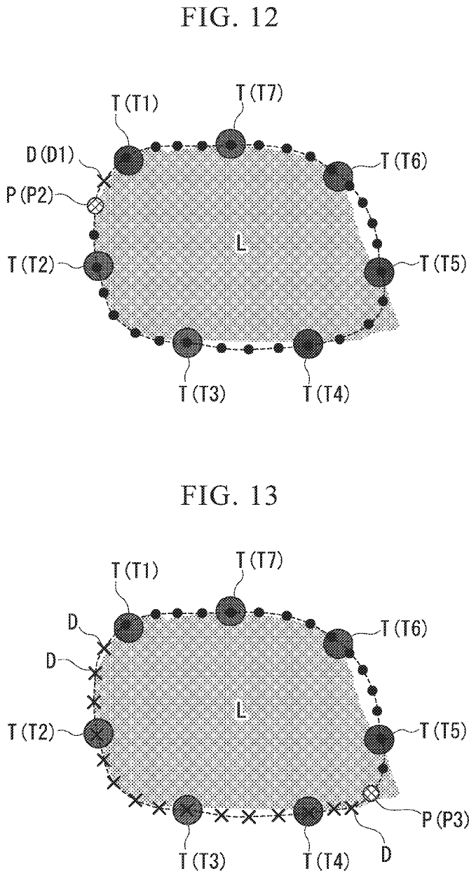

The operator Op supplies the electricity (Fire) to the electrical scalpel 48 in contact with the desired scheduled dissection point to cauterize the scheduled dissection point (treatment operation). Subsequently, as shown in FIG. 12, the cauterized point which is cauterized by the treatment operation is identified as “treatment point D”.

The detection unit 39 detects that the electricity is supplied (Fire) to the electrical scalpel 48, and the control unit 36 executes Step S27 after Step S25 and Step S26.

In Step S27, as shown in FIG. 17, in the same manner with that in Step S13, the control unit 36 records the treatment point D as a new teaching point T in the memory 36b and the storage 36c. That is, the treatment point D is recorded as the new teaching point T.

Subsequently, the control unit 36 executes Step S28.

In Step S28, as shown in FIG. 17, the control unit 36 determines whether the operation mode is the approach mode. In the situation in which the operation mode is still the approach mode, the control unit 36 executes Step S21.

In the situation in which the operation mode is not the approach mode, the control unit 36 executes Step S29 subsequently and the control of the approach mode is terminated.

The control unit 36 executes Step S21 again is configured to calculate the interpolation curve C again by taking the newly added teaching point T into consideration.

Subsequently, the control unit 36 executes Step S22.

The control unit 36 executes Step S22 again is configured to calculate the interpolation point P from the interpolation curve C that is calculated again.

As described above, in the approach mode, the treatment point D at which the treatment is actually performed is added as the new teaching point T such that the interpolation point P is updated accordingly. Thus, even in the situation in which necessary and enough teaching points T are not supplied in advance, the robot arm system 100 can prompt the interpolation points P reflecting the results of the dissection operation.

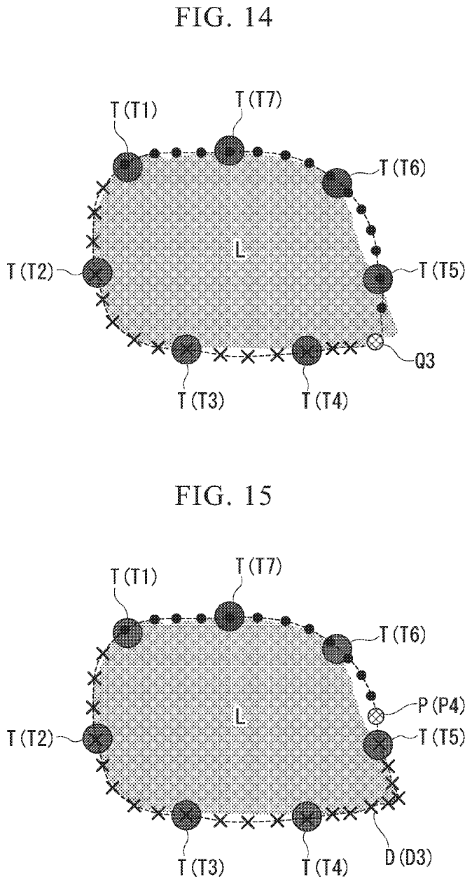

For example, it is assumed that the interpolation point P3 shown in FIG. 13 is inside the lesion portion L which is dissected and the interpolation point P3 is not the scheduled dissection point desired by the operator Op. The electrical scalpel 48 is moved to the interpolation point P3 by the control unit 36, however, the operator Op operates the operation arm 31 to move the electrical scalpel 48 to a desired scheduled dissection point Q3 around the surrounding of the lesion portion L, as shown in FIG. 14. Thereafter, the electrical scalpel 48 is supplied with the electricity (Fire), and the scheduled dissection point Q3 is marked as the treatment point D (D3). The operator Op moves the electrical scalpel 48 from the interpolation point P (P3) to the scheduled dissection point Q3 and then cauterizes the scheduled dissection point Q3 (treatment point D3).

Such an operation is repeated at each interpolation point, as shown in FIG. 15, the interpolation curve C approaches to the curve suitable surrounding the lesion portion L.

As shown in FIG. 13, in the situation in which the treatment points D increase, in Step S21, the teaching points T (the teaching points T2, T3, T4 in FIG. 13) through which the treatment points D pass may be removed from the teaching points T used for calculating the interpolation curve C. The treatment points D are more possible to be the scheduled dissection points desired by the operator Op with respect to the teaching points T such that the interpolation curve C can be revised to the interpolation curve desired by the operator Op.

After the dissection with respect to the surrounding of the lesion portion L, the operator Op or the scopist Sc terminates the control of the approach mode by the control unit 36 by changing the operation mode of the control unit 36 from the approach mode to another operation mode except the approach mode.

The control of the approach mode by the control unit 36 may be terminated by changing the operation mode of the control unit 36 from the approach mode to another operation mode except the approach mode during the period in which the electrical scalpel 48 is in standby for being supplied with the electricity (Fire) in Step S25 and Step S26.

### Effects According to First Embodiment

According to the robot arm system 100 according to the present embodiment, the treatment portion 41 is automatically moved to the interpolation points P by teaching the teaching points T to calculate the interpolation points P. The operation workload of the treatment tool unit 40 by the operator Op during the treatment procedures can be reduced.

According to the robot arm system 100 according to the present embodiment, even in the situation in which enough teaching points T are not prompted correctly in advance, during the procedures of the dissection treatment, the interpolation points reflecting the results of the dissection treatment can be prompted.

### MODIFICATION EXAMPLE

Although the preferred First Embodiment of the present invention has been described above by referring to figures, the present invention is not limited to the embodiment. Additions, omissions, substitutions and other changes in the structure are possible without departing from the spirit of the present invention.

For example, in the present embodiment, the robot arm system 100 can be applied to a medical robot arm, however, the robot arm system 100 is not exclusively limited to be used therein. For example, the robot arm system 100 can be applied in an industrial robot arm system. For example, it can be applied to the robot arm for welding used in an environment where enough teaching points are not supplied correctly in advance.

For example, in the present embodiment, the treatment points D are formed in a sequence from the initial treatment point D1 in the counterclockwise direction, the sequence of forming the treatment points D are not limited thereto. The treatment points D may be formed in the sequence in the clockwise direction. Furthermore, the treatment points D may be formed in an irregular sequence.

### Second Embodiment

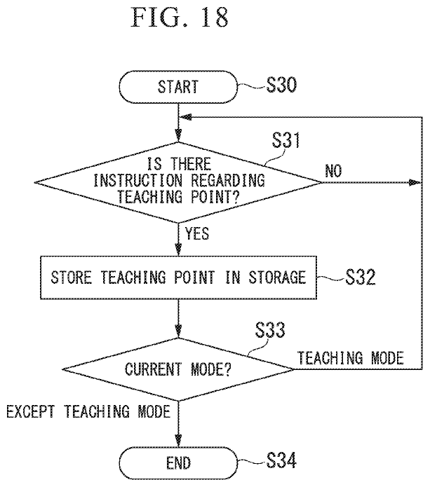

A second embodiment of the present invention will be described by referring to FIG. 18. In the present embodiment, an embodiment of prompting the teaching points T is different with that in the first embodiment. In the description below, the common configurations which has already described will be followed by the same reference sign and the reductant description will be omitted.

An overall configuration of a robot arm system 200 according to the present embodiment is same with that of the robot arm system 100 according to the first embodiment. Comparing with the robot arm system 100, in the robot arm system 200, the control unit 36 has an operation mode, specifically a teaching mode (first mode), instead of the marking mode.

The operation portion 30 of the robot arm system 200 further has an input device 29 such as a touch panel, a mouse, and the like, and the operator Op or the scopist Sc can assign the positions in the display screen of the monitor 22 by the input device 29. The position assigned in the display screen of the monitor 22 by the input device 29 is input to the control unit 36.

Until the procedures of operating the operation arm 31 by the operator Op and operating the endoscope 10 by the scopist Sc so as to move the treatment portion 41 and the endoscope 10 to the position where it is possible to perform the treatment with respect to the target portion, the procedures of the robot arm system 200 are same with that of the robot arm system 100 according to the first embodiment.

Thereafter, the control flow of the control unit 36 in the teaching mode will be described by referring to FIG. 18. FIG. 18 is a control flow chart of the control unit 36 in the teaching mode.

In the situation of performing the treatment to dissect the lesion portion by the operator Op, the operator Op or the scopist Sc sets the operation mode of the control unit 36 to the teaching mode.

As shown in FIG. 18, when the operation mode of the control unit 36 is changed to the teaching mode, the control unit 36 starts the control of the teaching mode (Step S30). Subsequently, the control unit 36 executes Step S31.

In the teaching mode, the operator Op does not operate the operation arm 31 to prompt the teaching points T. Instead, the operator Op prompts the teaching points T by using the input device 29 to assign the positions on the monitor 22 (teaching operation).

In Step S31, as shown in FIG. 18, the control unit 36 is in standby until there is an input from the input device 29 to teach the teaching points T. In the situation in which the teaching of the teaching points T are available by the input device 29, the control unit 36 executes Step S32 subsequently.

In Step S32, as shown in FIG. 18, the control unit 36 records the points prompted (taught, teaching operation) by the input device 29 in the memory 36b and the storage 36c as the teaching points T. The record targets are three-dimensional coordinates of the points taught by the input device 29. For example, if the endoscope is the configuration capable of capturing a stereo image, the three-dimensional coordinates of the prompted points can be calculated by calculating the three-dimensional coordinates from the stereo image.

In Step S32, the teaching points T that are prompted at once may be removed thereafter. The operator Op can perform a process of trial and error by repeatedly teaching and removing the teaching points T to determine the teaching point T.

The control unit 36 executes Step S33 subsequently.

In Step S33, as shown in FIG. 16, the control unit 36 determines whether the operation mode is the teaching mode. In the situation in which the operation mode is still the teaching mode, the control unit 36 executes Step S11 to be in standby for the teaching of the next teaching point.

In the situation in which the operation mode is not the teaching mode, the control unit 36 executes Step S34 subsequently and terminates the control of the teaching mode.

The operator Op or the scopist Sc can terminate the control of the teaching mode by the control unit 36 by changing the operation mode of the control unit 36 to another operation mode except the teaching mode.

Subsequently, the operator Op or the scopist Sc sets the operation mode of the control unit 36 to the approach mode, and dissects the lesion portion L in the same procedures with that of the robot arm system 100 according to the first embodiment.

### Effects According to Second Embodiment

According to the robot arm system 200 according to the present embodiment, together with the effects of the robot arm system 100 according to the first embodiment, the effects shown below can be achieved.

According to the robot arm system 200 according to the present embodiment, the operator Op can provide the teaching points T to the robot arm system 200 without actually performing the cauterization by the operator Op. Furthermore, according to the configuration capable of removing the teaching points T set at once, the operator can determine the teaching points T in a process of trial and error by repeatedly teaching and removing the teaching points T.

### Third Embodiment

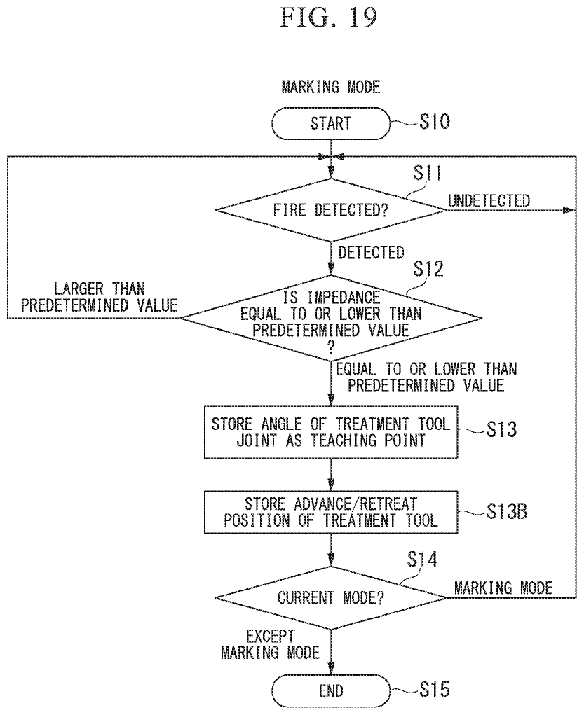

A third embodiment of the present invention will be described by referring to FIG. 19 to FIG. 21. In the present embodiment, an embodiment of storing the teaching points and the like are different with that according to the first embodiment.

An overall configuration of a robot arm system 300 according to the present embodiment is same with that of the robot arm system 100 according to the first embodiment.

Until the procedures of operating the operation arm 31 by the operator Op and operating the endoscope 10 by the scopist Sc so as to move the treatment portion 41 and the endoscope 10 to the position where it is possible to perform the treatment with respect to the target portion, the procedures of the robot arm system 200 are same with that of the robot arm system 100 according to the first embodiment.

Thereafter, the control flow of the control unit 36 in the marking mode will be described by referring to FIG. 19. FIG. 19 is a control flow chart of the control unit 36 in the marking mode.

Comparing with the control flow of the control unit 36 in the marking mode of the robot arm system 100 shown in FIG. 16, the control flow of the control unit 36 in the marking mode of the robot arm system 300 as shown in FIG. 19 is different only in that Step S13B is further included therein. The other steps are same with that according to the first embodiment and the description will be omitted.

In Step S13, as shown in FIG. 19, the control unit 36 records the point at which the electrical scalpel 48 is supplied with the electricity (Fire) as the teaching point T in the memory 36b and the storage 36c.

Subsequently, Step S13B is executed and the control unit 36 calculates the relative movement amount of the treatment tool unit 40 with respect to the console 21 from the relative movement amount of the base 32 with respect to the console 21. The control unit 36 records the relative movement amount of the treatment tool unit 40 together with the teaching point T to the memory 36b and the storage 36c.

Also, Step S13B can be executed in a replaced sequence with Step S13.

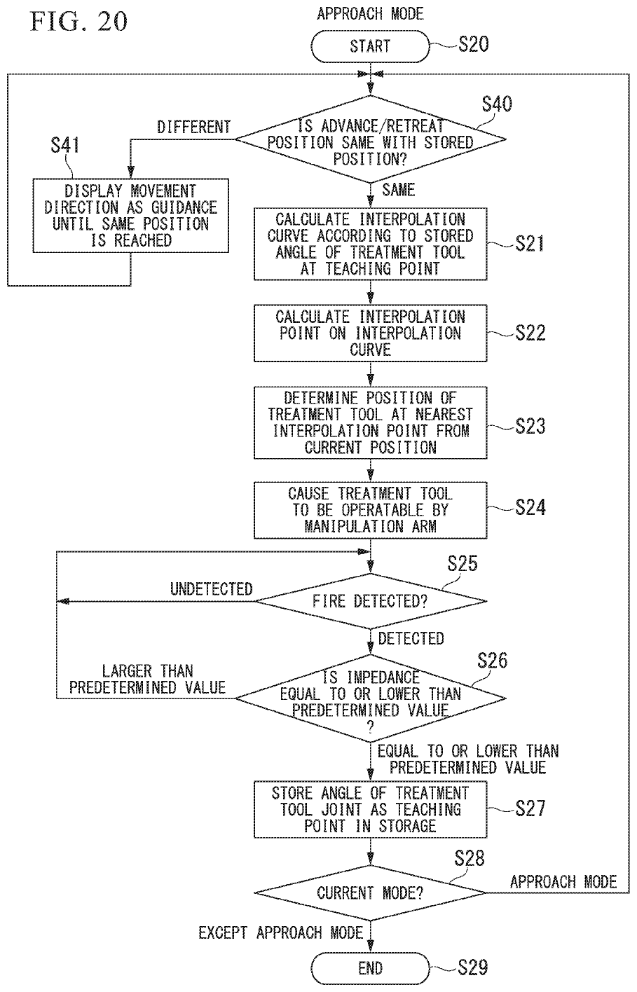

After the prompt of the teaching point T, the operator Op or the scopist Sc sets the operation mode of the control unit 36 to the approach mode. Hereinafter, the control flow of the control unit 36 in the approach mode will be described following the flow chart shown in FIG. 20.

Comparing with the control flow of the control unit 36 in the approach mode of the robot arm system 100 shown in FIG. 17, the control flow of the control unit 36 in the approach mode of the robot arm system 300 as shown in FIG. 20 is different only in that Step S40 and Step S41 are further included therein. The other steps are same and the description will be omitted.

As shown in FIG. 20, when the operation mode of the control unit 36 is changed to the approach mode, the control unit 36 starts the control of the approach mode (Step S20). Subsequently, the control unit 36 executes Step S40.

In Step S40, as shown in FIG. 20, the control unit 36 determines whether the relative movement amount of the treatment tool unit 40 with respect to the current console 21 is same with the relative movement amount of the treatment tool unit 40 stored together with the teaching point T in the marking mode.

In the situation in which the relative movement amount of the treatment tool unit 40 with respect to the current console 21 is same with the relative movement amount of the treatment tool unit 40 stored together with the teaching point T in the marking mode, the control unit 36 executes Step S21 subsequently. In the situation in which the relative movement amount of the treatment tool unit 40 with respect to the current console 21 is different from the relative movement amount of the treatment tool unit 40 stored together with the teaching point T in the marking mode, the control unit 36 executes Step S41 subsequently.

In Step S41, as shown in FIG. 20, the control unit 36 controls the monitor 22 to display a guidance to demand the operator Op to perform advancement and retraction of the treatment tool unit 40 so as to make the relative movement amount of the treatment tool unit 40 with respect to the current console 21 to be same with the relative movement amount of the treatment tool unit 40 stored together with the teaching point T in the marking mode. At this time, it is prefer to also display the direction and the movement amount for moving the base 32. Subsequently, the control unit 36 executes Step S40 again.

### Effects According to the Third Embodiment

According to the robot arm system 300 according to the present embodiment, together with the effects of the robot arm system 100 according to the first embodiment, the effects shown below can be achieved.

According to the robot arm system 300, in the approach mode, the control unit 36 can perform the automatic operation of the treatment tool unit 40 in consideration of the relative movement amount of the treatment tool unit 40 when the teaching points T are prompted to move the treatment portion 41 to the interpolation point P more accurately.

According to the robot arm system 300, the operator Op can perform other treatment such relatively moving the operation tool unit 40 to perform treatment with respect to other lesion portion between the treatment during the marking mode and the treatment during the approach mode.

### MODIFICATION EXAMPLE

Although the preferred Third Embodiment of the present invention has been described above referring to figures, the present invention is not limited to the embodiment. Additions, omissions, substitutions and other changes in the structure are possible without departing from the spirit of the present invention.

For example, in the above-described embodiment, the automatic operation of the treatment tool unit 40 after Step S21 is not performed in the situation in which the relative movement amount of the treatment tool unit 40 with respect to the current console 21 is not same with the relative movement amount of the treatment tool unit 40 stored together with the teaching point T.

However, even if the relative movement amount of the treatment tool unit 40 with respect to the current console 21 is different from the relative movement amount of the treatment tool unit 40 stored together with the teaching point T, there is a possibility that the treatment portion 41 of the treatment tool unit 40 can be determined and disposed at the position of the interpolation point P without the advancement and the retraction of the treatment tool unit 40. In this situation, it is not necessary to execute Step S41 to demand the operator Op to perform the advancement and retraction of the treatment tool unit 40.

Thus, in the control flow of the control unit 36 in the approach mode according to a modification example of the robot arm system 300, Step S40 and Step S41 are not executed.

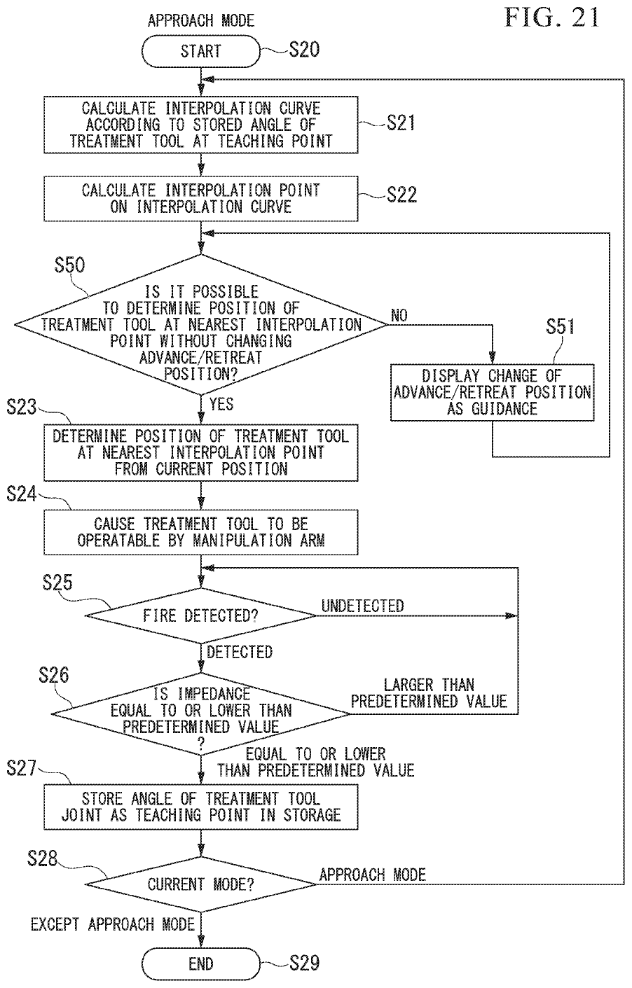

The control flow of the control unit 36 in the approach mode according to the modification will be described by referring to FIG. 21.

Comparing with the control flow of the control unit 36 in the approach mode of the robot arm system 100 shown in FIG. 17, the control flow of the control unit 36 in the approach mode according to the modification example of the robot arm system 300 as shown in FIG. 21 is different only in that Step S50 and Step S51 are further included therein. The other steps are same and the description will be omitted.

As shown in FIG. 20, when the operation mode of the control unit 36 is changed to the approach mode, the control unit 36 starts the control of the approach mode (Step S20). Subsequently, the control unit 36 executes Step S21.

After Step s22, the control unit 36 executes Step S50.

In Step s22, as shown in FIG. 21, the control unit 36 determines whether it is possible to drive the arm 42 to determine and dispose the treatment portion 41 at the nearest interpolation point P with respect to the treatment potion 41 among the plurality of interpolation points P calculated in Step S22 without changing the relative movement amount to change the advance/retract position of the treatment tool unit 40.

In the situation in which the control unit 36 determines that it is possible to dispose the treatment portion 41 at the nearest interpolation point P, the control unit 36 does not demand the advancement and the retraction of the treatment tool unit 40 and the control unit 36 executes Step S23.

In the situation in which the control unit 36 determines that it is impossible to dispose the treatment portion 41 at the nearest interpolation point P, the control unit 36 executes Step S51.

In Step S51, as shown in FIG. 21, in the same manner with that in Step S41, the control unit 36 controls the monitor 22 to display the guidance to demand the operator Op to perform advancement and retraction of the treatment tool unit 40. The control unit 36 executes Step S50 again.

As described above, it is possible to reduce the number of displaying the guidance on the monitor 22 and reduce the operation workload due to the advancement and retraction of the treatment tool unit 40 by the operator Op during the treatment.

Although preferred embodiments of the present invention have been described above, the present invention is not limited to the embodiments and modifications thereof. Additions, omissions, substitutions and other changes in the structure are possible without departing from the spirit of the present invention. The present invention is not limited by the foregoing description but is limited only by the scope of the appended claims.

What is claimed is:

1. A robot arm controller configured to control a treatment tool, the treatment tool having a robot arm with an electrically driven joint, the robot arm controller comprising:
a driver configured to drive the treatment tool;
a manipulator configured to accept a manipulation input for the treatment tool; and
a processor configured to control the treatment tool,
wherein the processor is configured to:
store a position of a distal end portion of the treatment tool as a teaching point when a teaching operation is detected from the manipulator,
calculate an interpolation curve passing a plurality of teaching points and an interpolation point on the interpolation curve,
control the driver to move the distal end portion of the treatment tool to the interpolation point, and
store the position of the distal end portion of the treatment tool as a treatment point when a treatment operation is detected from the manipulator, and
wherein the interpolation curve is updated so as to pass the plurality of teaching points and the treatment point.

2. The robot arm controller according to claim 1,
wherein the processor is configured to be operated under a selected operation mode between a first mode and a second mode,
wherein the processor is configured to detect the teaching operation from the manipulator in response to selecting the first mode, and
wherein the processor is configured to calculate the interpolation point and control the distal end portion of the treatment tool to the interpolation point in response to selecting the second mode.

3. The robot arm controller according to claim 1, wherein the processor is configured to cause the position of the distal end portion of the treatment tool to be adjustable according to the manipulation input from the manipulator while controlling the driver to move the distal end portion of the treatment tool to the interpolation point.

4. A robot system, comprising:
a treatment tool having a robot arm with an electrically driven joint;
a console having the robot arm controller according to claim 1; and
an endoscope.

5. The robot system according to claim 4,
wherein the treatment tool is attached to the console to be relative movable with respect to the console,
wherein the manipulator is configured to be able to make the treatment tool to relatively move with respect to the console, and
wherein the processor is configured to store a relative movement amount of the treatment tool with respect to the console used while storing the teaching point and the processor is configured to use the relative movement amount to calculate the interpolation point.
